SEQUENCE LISTING (10 sequences)

SEQ ID NO: 1 - protein, 855 aa, synthetic construct
EFDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLNSAVDHHHHHH

SEQ ID NO: 2 - protein, 252 aa, synthetic construct
EFEMQLVESGGGLVQPGRSMKLSCAGSGFTLSDYGVAWVRQAPKKGLEWVAYISYAGGTTYYRESVKGRFTISRDNAKSTLYLQMDSLRSEDTATYYCTIDGYGGYSGSHWYFDFWGPGTMVTVSSGGGGSGGGGSGGGGSDIQMTQSPSLLSASVGDRVTLNCRTSQNVYKNLAWYQQQLGEAPKLLIYNANSLQAGIPSRFSGSGSGTDFTLTISSLQPEDVATYFCQQYYSGNTFGAGTNLELKGGSGG

SEQ ID NO: 3 - protein, 861 aa, synthetic construct
EFDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGSGGAAARGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALANLEQKLISEEDLNSAVDHHHHHH

SEQ ID NO: 4 - protein, 857 aa, synthetic construct
EFSYVLTQPPSVSVAPGQTARITCGGNNIGSKSVHWYQQKPGQAPVLVVYDDNDRPSGLPERFSGSNSGNTATLTISRVEAGDEADYYCQVWDSSSDHVVFGGGTKLTVLGGGGSGGGGSGGGGSEVQLLEPGGGLVQPGGSLRLSCEASGSTFSTYAMSWVRQAPGKGLEWVSGFSGSGGFTFYADSVRGRFTISRDSSKNTLFLQMSSLRAEDTAVYYCAIPARGYNYGSFQHWGQGTLVTVSSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLNSAVDHHHHHH

SEQ ID NO: 5 - protein, 242 aa, synthetic construct
EFQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSGGGGSGGGGSGGGGSEIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKGGSGG

SEQ ID NO: 6 - protein, 867 aa, synthetic construct
EFDIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKGGGGSGGGGSGGGGSEVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLEQKLISEEDLNSAVDHHHHHH

SEQ ID NO: 7 - DNA, 19 bp, synthetic construct
  source 1..19: mol_type = other DNA
taatacgactcactatagg

SEQ ID NO: 8 - DNA, 18 bp, synthetic construct
  source 1..18: mol_type = other DNA
tagaaggcacagtcgagg

SEQ ID NO: 9 - DNA, 39 bp, synthetic construct
  source 1..39: mol_type = other DNA
gtccccagtcatcagctcctaggggtgtgtttcgccgag

SEQ ID NO: 10 - DNA, 30 bp, synthetic construct
  source 1..30: mol_type = other DNA
gactctagaggctaaggcgtctttgcatct

(19) United States
(12) Patent Application Publication
(10) Pub. No.: US 2024/0002485 A1
(43) Pub. Date: Jan. 4, 2024

(54) FUSION PROTEIN BASED ON SINGLE-CHAIN ANTIBODY FRAGMENT, NANO-ASSEMBLY, AND PREPARATION METHODS THEREFOR AND APPLICATIONS THEREOF

(71) Applicant: SOUTH CHINA UNIVERSITY OF TECHNOLOGY, Guangdong (CN)

(72) Inventors: Jun WANG, Guangdong (CN); Song SHEN, Guangdong (CN); Dongkun ZHAO, Guangdong (CN)

(73) Assignee: SOUTH CHINA UNIVERSITY OF TECHNOLOGY, Guangdong (CN)

(21) Appl. No.: 18/365,239

(22) Filed: Aug. 4, 2023

Related U.S. Application Data
(63) Continuation of application No. PCT/CN22/74716, filed on Jan. 28, 2022.

(30) Foreign Application Priority Data
Feb. 5, 2021 (CN) 202110164375.1

(51) Int. Cl. C07K 16/18; C08L 67/04; B82Y 5/00

(52) U.S. Cl. CPC C07K 16/18; C08L 67/04; B82Y 5/00; C07K 2319/31; C07K 2317/622

(57) ABSTRACT

A recombinant fusion protein, a nano-assembly, and preparation methods therefor and the use thereof. The nano-assembly is constructed from at least one recombinant fusion protein and a hydrophobic degradable polyester and a derivative thereof. The recombinant fusion protein comprises a single-chain antibody fragment, a linker peptide and an albumin having a hydrophobic region, wherein the single-chain antibody fragment has at least one of the effects of competitively binding to a target, inhibiting a signaling pathway, activating a signaling pathway, and targeting an antigen. The present disclosure further relates to a method for preparing a monoclonal antibody fragment-albumin recombinant fusion protein, and a method for constructing a nano-assembly. On the basis of the nano-assembly, a brand-new method for constructing a mono-specific, bispecific or multispecific antibody is provided, and the nano-assembly can be used for treating diseases such as tumors and autoimmune diseases.

### CROSS-REFERENCE TO RELATED APPLICATION

This application is a continuation application of international application PCT application serial no. PCT/CN2022/074716, filed on Jan. 28, 2022, which claims the priority benefit of China application no. 202110164375.1, filed on Feb. 5, 2021. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jul. 27, 2023, is named 136168_SEQUENCELISTING and is 13,800 bytes in size.

### BACKGROUND OF THE INVENTION

### 1. Technical Field

The present disclosure relates to the technical field of pharmaceutical technology, in particular to a fusion protein and nano-assembly based on single-chain antibody fragment, preparation method and applications thereof.

### 2. Background Art

Monoclonal antibody drugs represented by immune checkpoint blocking antibodies have become a new hotspot in the global biopharmaceutical field. PD1/PDL1 and CTLA4 monoclonal antibody drugs that have been marketed are profoundly changing the pattern of tumor treatment. At present, more than ten kinds of PD1/PDL1 monoclonal antibody drugs have been approved for the treatment of different types of tumors such as non-small cell lung cancer and melanoma in China. At the same time, the research and development of monoclonal antibody drugs targeting other immune checkpoint molecules of T cells (TIGIT, OX40, LAG3, TIM3, etc.) and other immune cells (including macrophages, natural killer cell, etc.) are also in full swing.

Immune checkpoint monoclonal antibodies have shown enormous commercial value and broad clinical application prospects, but different types of tumors and different patients with the same type of tumors have very different responses to this type of immunotherapy, and the overall clinical response rate is low. In recent years, bispecific antibodies and even multispecific antibodies have attracted increasing attention as an effective strategy and have been developed to overcome the problem of insufficient potency of monoclonal antibody drugs. For example, Blinatumomab, approved in 2014 by the U.S. Food and Drug Administration for the treatment of B-cell precursor acute lymphocytic leukemia, is a CD19×CD3 bispecific antibody that can simultaneously bind to CD19 molecules on the surface of B lymphoblasts and CD3 molecules on the surface of T cells, activating T cells and bridging the two types of cells at the same time, thereby enhancing the killing of tumor cells by T cells. Studies have shown that trispecific antibodies have stronger tumor recognition and killing ability than bispecific antibodies and monoclonal antibodies. For example, the CD3×CD28×CD38 trispecific antibody developed by Sanofi (France) not only has the ability of CD3×CD38 bispecific antibody to bridge effector-target cells and activate T cells, but also provides activation and co-stimulatory signals at the same time, preventing T cell apoptosis, and exerts a stronger anti-tumor effect. Although bispecific/multispecific antibodies are expected to improve the antibody titers and tumor treatment effects, their structural design and production process are significantly more complex compared with monoclonal antibodies. Taking the preparation of bispecific antibody by genetic recombination technology as an example, theoretically, more than ten kinds of by-products will be produced when expressing bispecific antibody. Although researchers have developed molecular design strategies to reduce the production of certain by-products, they cannot completely eliminate all of them, which presents significant challenges to downstream process development.

Nanoparticles, as a kind of efficient drug delivery carriers, are widely used to improve the metabolic behavior and efficacy of small molecule drugs, protein drugs, and nucleic acid drugs. Nanocarriers have shown unique advantages in the delivery of antibody drugs such as blocking antibodies at immune checkpoints. It is particularly important to point out that immobilizing two or more monoclonal antibody drugs on the surface of nanoparticles can not only achieve multispecificity of antibody drugs, but also achieve “multivalent states” (multiple antibody molecules are immobilized on the same particle surface), which may be an important reason for the more efficient anti-tumor effects of “carrier based” antibody drugs.

As mentioned above, attaching multiple monoclonal antibody drugs to the surface of nanocarriers is a potential strategy for improving antibody efficacy. However, the reported methods of immobilizing antibodies are mainly to bond the amino, carboxyl, thiol groups and other functional groups on antibody drug molecules to the surface of particles, but these methods have many disadvantages, such as complex reaction and purification processes, and damaging the advanced structure of antibodies or blocking the antigen recognition region of therapeutic antibodies, and significantly reducing the ability of antibodies to recognize antigens, etc.

A single-chain variable fragment (scFv) refers to a brief part of an immunoglobulin Fab fragment that is artificially expressed and retained by antigen-specific binding through gene recombination technology. It consists of a heavy chain and a light chain, and the two chains are connected by a short polypeptide hinge. scFv not only retains the characteristics of antigen recognition and binding, but also has small molecules, which can be recombined with polypeptides or proteins to form fusion proteins. Serum albumin and other proteins are widely used in the construction of drug delivery carriers, and drug loaded albumin nanoparticles such as Abraxane have been approved for the treatment of malignant tumors. If scFv is combined with serum albumin to form a fusion protein, and two or more fusion proteins are assembled together, a new type of double/multi specific antibody can be constructed, which has the characteristics of multispecificity and multivalence, and is expected to play an important role in the treatment of tumors and other diseases.

### SUMMARY OF THE INVENTION

Based on the above, one of the purposes of the present disclosure is to provide a recombinant fusion protein, which can construct a nano-assembly that can be used for the delivery of at least one kind of single-chain antibody fragment, exerting the functions of monospecific, bispecific, or multispecific antibodies.

A class of recombinant fusion protein, including a protein with a hydrophobic region and an immunoregulatory antibody functional fragment, wherein the protein with hydrophobic region and the immunoregulatory antibody functional fragment are directly connected or connected through peptide connectors; the immunoregulatory antibody functional fragment is a single-chain antibody fragment, and the single-chain antibody fragment has the ability to specifically recognize and bind to an antigen.

The second purpose of the present disclosure is to provide a nano-assembly for delivering at least one single-chain antibody fragment.

The nano-assembly is formed by combining at least one of the above-mentioned fusion proteins and a hydrophobic degradable polyester or its derivatives through hydrophobic interactions, and the single-chain antibody fragments of different fusion proteins are different.

The third purpose of the present disclosure is to provide a preparation method for the above-mentioned nano-assembly which comprises the following steps:
(1) mixing the above-mentioned fusion protein with water or an aqueous solution to obtain a water phase with a concentration of 0.5 mg/mL to 20 mg/mL, preferably 5 mg/mL to 10 mg/mL; and
mixing the hydrophobic degradable polyester and its derivatives with an organic solvent at a concentration of 0.5 mg/mL to 10 mg/mL, preferably in the range from 1 mg/mL to 5 mg/mL, to obtain an oil phase; and
(2) preparing the water phase and oil phase in step (1) into an oil-in-water emulsion, preferably the volume ratio of the water phase to the oil phase is 1:1 to 10:1, preferably 5:1 to 10:1; and
(3) separating and purifying the emulsion to obtain a nano-assembly.

The fourth purpose of the present disclosure is to provide an application of the above-mentioned fusion protein in the preparation of the nano-assembly for delivering at least one single-chain antibody fragment.

Another purpose of the present disclosure is to provide an application of the above-mentioned nano-assembly in the preparation of therapeutic immune drugs.

Compared with the prior art, the present disclosure has the following beneficial effects:

In the present disclosure, hydrophobic degradable polyester or other hydrophobic molecules and the recombinant fusion protein of specific protein with hydrophobic domain is selected to form a multivalent nano-assembly capable of targeting a single antigenic epitope or multiple antigenic epitope. This method can quickly and stably prepare nano-assemblies containing different types and different ratios of single-chain antibody fragments by simple physical mixing and adjusting the mixing ratio of different recombinant fusion proteins, which conveniently achieves the “multivalent effect” and “multispecific effect” of single-chain antibody fragments.

The nano-assembly obtained in the present disclosure is formed by entanglement and assembly of hydrophobic polyester or other hydrophobic molecules with the hydrophobic domain of the protein through hydrophobic interactions. It has excellent stability, and the activities between single-chain antibody fragments are not affected by each other.

The present disclosure uses this constructed nano-assembly for the first time in the development of drugs for tumors or autoimmune diseases.

### BRIEF DESCRIPTION OF THE DRAWINGS

FIG. 1 shows nucleic acid gel electrophoresis of the fragment of mouse MSA gene obtained by PCR.

FIG. 2 shows the results of nucleic acid gel electrophoresis for identification of recombinant plasmid transformed into Escherichia coli by PCR method.

FIG. 3 shows the results of screening Yeast Mutt recombinants using selective culture medium, with Yeast Mut+ marked as a check mark.

FIG. 4 shows nucleic acid gel electrophoresis identification of yeast recombinants selected by PCR.

FIG. 5 shows the identification of recombinant fusion proteins expressed in recombinant yeast cells through SDS-PAGE.

FIG. 6 shows the identification of recombinant fusion proteins expressed in recombinant yeast cells through the Western Blot method.

FIG. 7 shows the identification of recombinant fusion proteins expressed in mammalian cell systems through SDS-PAGE.

FIG. 8 shows the identification of recombinant fusion proteins expressed in mammalian cell systems through the Western Blot method.

FIG. 9 shows the identification of the biological activity of recombinant fusion proteins expressed in mammalian cell or yeast systems through ELISA.

FIG. 10 shows the particle size of nano-assembly prepared with recombinant proteins using Zetasizer.

FIG. 11 shows the morphology characterization of the recombinant fusion protein nano-assembly.

FIG. 12 shows the effect of αPD1/α4-1BB nano-assembly on activating T cells proliferation.

FIG. 13 shows the effect of αPD1/α4-1BB nano-assembly on maintaining T cells survival.

FIG. 14 shows the effect of αPD1/α4-1BB nano-assembly enhancing T cells specific killing of tumor cells.

FIG. 15 shows the effect of αPD1/α4-1BB nano-assembly on enhancing cytokine IFN-γ secretion in T cells.

FIG. 16 is a schematic diagram of the principles of the fusion protein, assembly, and application of the present disclosure.

### DETAILED DESCRIPTION OF THE EMBODIMENTS

In the following embodiments of the present disclosure, the experimental methods without specific conditions are generally in accordance with conventional conditions or in accordance with the conditions recommended by the manufacturers. Various common chemical reagents used in the examples are all commercially available products.

Unless otherwise defined, all technical and scientific terms used in the present disclosure are the same as commonly understood by those skilled in the art. The terms used in the specification of the present disclosure are only for the purpose of describing specific embodiments, but not used to limit the present disclosure.

The terms “including” and “having” and any variations thereof in the present disclosure are intended to cover non-exclusive inclusions. For example, a process, method, device, product, or equipment which includes a series of steps is not limited to the listed steps or modules, but optionally includes steps that are not listed, or optionally other steps which are includes inherently in those processes, method, product, or equipment.

The “plurality” mentioned in the present disclosure means two or more. “And/or” describes the association relationship of the associated objects, indicating that there can be three types of relationships, for example, A and/or B, which can mean: A exists alone, A and B exist at the same time, and B exists alone. The character “/” generally indicates that the associated objects before and after are in an “or” relationship.

The embodiments of the present disclosure relate to a fusion protein, including a protein with a hydrophobic region, a connecting peptide, and a single-chain antibody fragment.

In some embodiments, the types of single-chain antibody fragments include but not limited to single-chain antibody fragments derived from the following antibodies of species such as humans, mice, or rats: CD137L, CD137, 4-1BBL, 4-1BB, OX40L, OX40, ICOSL, ICOS, CD86, CD80, CD28, LFA3, CD2, CTLA4, PDL1, PD1, CD70, CD27, GAL9, TIM3, CD111, CD96, CD112, CD226, CD115, CD113, TIGIT, CD39, CD73, CD47, SIRPα, TNFα, IL1β, IL6, TGFβ, IL-10, IL-12, so correspondingly, the fusion protein can target the site of CD137L, CD137, 4-1BBL, 4-1BB, OX40L, OX40, ICOSL, ICOS, CD86, CD80, CD28, LFA3, CD2, CTLA4, PDL1, PD1, CD70, CD27, GAL9, TIM3, CD111, CD96, CD112, CD226, CD115, CD113, TIGIT, CD39, CD73, CD47, SIRPα, TNFα, IL1β, IL6, TGFβ, IL-10, IL-12, thus having the ability to competitively bind targets, inhibit signaling pathways, activate signaling pathways, and target antigens.

In some embodiments, the single-chain antibody fragments are formed by connecting the antibody heavy chain variable region VH and the antibody light chain variable region VL, and the connection configuration includes at least one of the following: VL-VH-AC, VL-VH-AN, VH-VL-AC, VH-VL-AN, VL-VH-L-AC, VL-VH-L-AN, VH-VL-L-AC, VH-VL-L-AN, wherein VL is the variable region of the antibody light chain, peptide or polypeptide sequence; VH is the variable region of the antibody heavy chain, peptide or polypeptide sequence, therapeutic protein and its fragments; L is the connecting peptide; AC is the C-terminal end of the serum albumin sequence, and AN is the N-terminal end of the serum albumin sequence. The VH is connected to VL through a connecting peptide, which can be at least one of the connecting peptides including but not limited to (GGGGS)n, (EAAAK)n, wherein n is any integer.

In some embodiments, the protein has at least three hydrophobic regions, which can combine with hydrophobic degradable and its derivatives through hydrophobic interactions. In the present disclosure, it is albumin, as known as serum albumin, which can be at least one from human serum albumin, bovine serum albumin, mouse serum albumin, rat serum albumin, rabbit serum albumin, chicken egg albumin, immunoglobulin G, protein A and protein G.

In some preferred embodiments, the protein is derived from human serum albumin with at least three or four hydrophobic domains. The protein with hydrophobic domains is derived from human serum albumin with at least five hydrophobic domains, or above-mentioned proteins with complete hydrophobic domains through substitution, deletion, and/or addition of one or more amino acids; more preferably, proteins from human serum albumin with six or seven hydrophobic domains.

The connecting peptide (peptide connector) can be a peptide segment sequence commonly used to connect polypeptides, which can connect two polypeptides and naturally fold them into the desired structure. Typically, it is a short peptide with hydrophobicity and certain extensibility. In the present disclosure, the purpose is to separate the two fused proteins to alleviate their mutual interference. The peptide connector can be flexible or rigid. In some embodiments, flexible peptide connectors may be advantageous as they can connect two protein/polypeptide components while maintaining their respective activity and function. In some embodiments, in the fusion protein, the protein in the hydrophobic region and the functional fragment of the immune regulatory antibody are connected by a peptide connector. In some embodiments, for example, [GlyGlyGlyGlySer]n is used, wherein n is an integer of 0-4, more preferably 1, 2, and 3. When n is 0, it means that the protein in the hydrophobic region is directly connected to the immune regulatory antibody functional fragment (single-chain antibody fragment).

In some embodiments of the present disclosure, gene vectors for recombinant fusion proteins based on single-chain antibody fragments include but are not limited to: pcDNA3.1, pcDNA3.0, pcDNA2.0, pcDNA4.0, pPICZa, pPICZb, pPICZc vector plasmids, and other similar gene vectors that can be expressed in yeast or mammalian cells, insect cells, and in vitro expression systems.

The scFv single-chain antibody fragment and its variant sequence can be formed by the antibody light chain variable region and the antibody heavy chain variable region of the respectively queried from the well-known database IMGT in the field and connected by connecting peptides, wherein the antibody light chain variable region and the antibody heavy chain variable region sequences can be from the light chain variable region and heavy chain variable region of the same antibody, or they can be combined from the light chain variable region and heavy chain variable region of different antibodies, or from the heavy chain variable region of a single-domain antibody.

In some embodiments of the present disclosure, genes encoding the above-mentioned recombinant fusion protein, or plasmid vectors containing the genes encoding the above-mentioned recombinant fusion protein are involved.

In some embodiments, common methods can be used to convert/transfect the desired nucleic acid into the host cell, such as electroporation, liposome transfection, PEI transfection, lentivirus transfection, preparation of receptive protoplasts, etc.

In some embodiments, the cells successfully transformed/transfected, i.e. the cells containing the gene construct of the present disclosure, can be identified by techniques well-known in the field, such as extracting genomic DNA from lysis cells for PCR reaction identification, or using immunoaffinity techniques such as Western Blot and ELISA to identify recombinant fusion protein His tags and HSA, MSA, and single-chain antibody fragments kappa chains in cells or cell culture supernatants.

In some embodiments, the fusion protein of the present disclosure can be produced by cultivating hosts containing the gene construct of the present disclosure, such as recombinant yeast, recombinant mammalian cells, recombinant insect cells, recombinant bacteria, transgenic animals and plants, etc. The specific cultivation methods can be used, such as shaking flasks, rotating bottles, bioreactors, etc.

In some embodiments the recombinant fusion protein can exist inside the host cell or can be secreted from the host, preferably be secreted from the host. The signal peptides used for secretion are preferably yeast α-factor signal peptide and natural IL-2 signal peptide sequence, or the analog of these two signal peptides. The gene encoding the fusion protein can be inserted into the host chromosome or exist as an episomal plasmid

In some embodiments, the culture medium or cells containing recombinant fusion proteins can be collected by centrifugation. The isolation, purification and concentration of the recombinant fusion protein can use common technologies in the art, such as salting out, organic reagent precipitation, isoelectric point precipitation, ultrafiltration, dialysis, tangential flow, liquid chromatography, and combinations of these technologies, wherein liquid chromatography can use gel exclusion, affinity, ion exchange, hydrophobic, reverse phase chromatography etc.

The above-mentioned recombinant fusion protein can be used to prepare a nano-assembly for delivery of at least one single-chain antibody fragment.

Some embodiments of the present disclosure relate to a nano-assembly. The nano-assembly is formed by combing at least one of the above-mentioned fusion proteins with hydrophobic degradable polyester and its derivatives through hydrophobic interactions the single-chain antibody fragments of different fusion proteins are different.

The nano-assembly can be composed of multiple recombinant fusion proteins and hydrophobic materials. The single-chain antibody fragments in each different recombinant fusion protein must be different, but the proteins with hydrophobic regions that form the fusion protein can be the same or different. Such nano-assembly is used to deliver different single-chain antibody fragments for recognition and binding with different antigens.

Another component of the nano-assembly is hydrophobic polymer materials, including but not limited to: hydrophobic aliphatic polyesters with poor water affinity, long-chain fatty acids, and/or hydrophobic small molecule drugs.

The hydrophobic degradable polyester and its derivatives can be currently known biodegradable biomaterials, as well as new degradable biomaterials that will be developed in the future, which can combine with the hydrophobic regions of the protein portion in the fusion protein. The polyester is an aliphatic polyester or its derivative, or polyethylene glycol modified aliphatic polyester or its derivatives.

The hydrophobic aliphatic polyester is an aliphatic polyester and/or a derivative of aliphatic polyester modified by different side and end groups, at least one of the polyethylene glycol modified aliphatic polyester and/or a derivative modified by different side and end groups, and has biocompatibility and biodegradability.

The aliphatic polyester includes but is not limited to at least one of the following: polylactide, polyglycolide, poly(lactide-co-glycolide) and polycaprolactone; the polyethylene glycol modified aliphatic polyester includes but is not limited to at least one of the following: polyethylene glycol modified polylactide, polyethylene glycol modified polyglycolide, polyethylene glycol modified poly(lactide-co-glycolide) and polyethylene glycol modified polycaprolactone.

The aliphatic polyester is polylactide, which is poly(L-lactide), poly(R-lactide), or a racemic polylactide; the end group of the polylactide is at least one of: ester group, carboxyl group, or hydroxyl group.

In the hydrophobic material, the ratio range of LA/GA in poly (lactide-co-glycolide) is from 95/5 to 50/50.

The polylactide is a levorotatory polylactide, and the end group of the levorotatory polylactide is an ester group. The molecular weight range of the levorotatory polylactide is 7200 Daltons to 1100000 Daltons, preferably 137000 Daltons to 240000 Daltons.

In some embodiments, the nano-assembly is a nanoparticle with a particle size ranges from 20 nm to 400 nm, preferably 80 nm to 200 nm, and preferably 80 nm to 150 nm.

Some embodiments of the present disclosure relate to a preparation method for the above-mentioned nano-assembly comprising the following steps:
(1) mixing the fusion protein with water or an aqueous solution to obtain a water phase; mixing the hydrophobic degradable polyester and its derivatives with an organic solvent to obtain an oil phase;
(2) preparing the water phase and oil phase in step (1) into an oil-in-water emulsion;
(3) separating and purifying the emulsion to obtain a nano-assembly.

In some embodiments, oil-in-water emulsions are prepared by using microfluidics, high-pressure homogenization, ultrasound, and other methods;
or, the emulsion can be separated and purified by rotary evaporation, freeze-drying, centrifugation, chromatography, ultra-filtration, and other methods to obtain a nano-assembly.

In some embodiments, the concentration of the fusion protein in the water phase is 0.5 mg/mL to 20 mg/mL, preferably 5 mg/mL to 10 mg/mL; the concentration of hydrophobic degradable polyester and its derivatives in the oil phase is 0.5 mg/mL to 10 mg/mL, preferably 1 mg/mL to 5 mg/mL.

The volume ratio of the water phase to the oil phase is 1:1 to 10:1, preferably 5:1 to 10:1.

In some embodiments, the weight ratio of the polyester to protein is 1:0.1 to 1:30, preferably 1:1 to 1:25, preferably 1:2 to 1:15, and more preferably 1:3 to 1:15.

The solvent of the hydrophobic material is at least one of the following: chloroform, dichloromethane, ethyl acetate, methanol, and acetonitrile, preferably dichloromethane, ethyl acetate, methanol, more preferably dichloromethane, ethyl acetate.

In some embodiments, the above-mentioned nanoparticles are prepared without additional stabilizers.

In some embodiments, nanoparticles can be separated from free proteins by at least one of the following methods: centrifugation, tangential flow dialysis (dialysis under tangential shear force through a tangential flow device), and exclusion chromatography (based on the molecular weight of nanoparticles and free proteins).

In some embodiments, the method of preparing the water phase and the oil phases into an oil-in-water emulsion includes: ultrasonic emulsification, high-pressure homogenization emulsification, or microfluidic control.

The concentration of the recombinant fusion protein in the water phase is 0.5 mg/mL to 20 mg/mL, preferably 5 mg/mL to 10 mg/mL; the concentration of the polyester in the oil phase is 0.5 mg/mL to 10 mg/mL preferably 1 mg/mL to 5 mg/mL.

Some embodiments of the present disclosure relate to an application of the above-mentioned nano-assembly as an anti-tumor therapeutic drug.

In some embodiments, the anti-tumor therapeutic drugs are tumor immunotherapeutic drugs or tumor targeted therapy drugs.

In some embodiments, the nano-assembly of the present disclosure can be assembled from FDA approved polymer polyester and albumin fusion proteins, which exhibits excellent biocompatibility.

In some embodiments, the nano-assembly can simultaneously carry multiple recombinant fusion proteins, including but not limited to two or more types of αHER2 scFv-MSA, α4-1BB scFv-MSA, αCD19 scFv-HSA, αPDL1 scFv-HSA, αTIGIT scFv-HSA, αPD1 scFv-HSA.

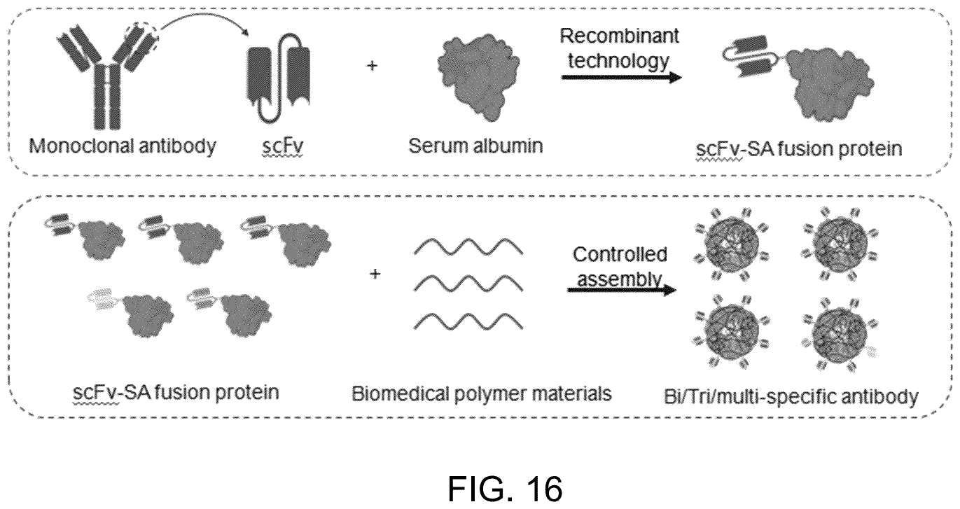

The nano-assembly of single-chain antibody fragment recombinant fusion protein described in the present disclosure is assembled through the hydrophobic interaction between polylactic acid and the albumin structure in the recombinant fusion protein, forming a nano-assembly with a stable structure, which can carry a variety of single-chain antibody fragments targeting the same or different targets. The mild assembly conditions will not destroy the structure of the antigen recognition domain (FIG. 16). It overcomes the defects of traditional chemical bond fixation methods, such as destroying the structure of antibody drugs, blocking the antibody recognition regions, significantly affecting the function of antibody drugs, high complexity and difficulty, etc, providing a new and simple structural design for the development of combined antibody therapy.

The nano-assembly of the present disclosure can be assembled from FDA approved polyester and natural serum albumin, exhibiting excellent biocompatibility.

The following will provide a further detailed explanation of the present disclosure in conjunction with specific embodiments.

The following are the relevant sequences used in the embodiments.

SEQ ID No. 1:
αHer2 scFv-HSA
EFDIQMTQSPSSLSASVGDRVTITCRASQDVNTAVAWYQQKPGKAPKLLIYSASFL
YSGVPSRFSGSRSGTDFTLTISSLOPEDFATYYCQQHYTTPPTFGQGTKVEIKGGGGSGG
GGSGGGGSEVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWGGDGFYAM
DYWGQGTLVTVSSGGGGSDAHKSEVAHRFKDLGEENFKALVLIAFAQYLQQCPFEDH
VKLVNEVTEFAKTCVADESAENCDKSLHTLFGDKLCTVATLRETYGEMADCCAKQEPE
RNECFLQHKDDNPNLPRLVRPEVDVMCTAFHDNEETFLKKYLYEIARRHPYFYAPELLF
FAKRYKAAFTECCQAADKAACLLPKLDELRDEGKASSAKQRLKCASLQKFGERAFKA
WAVARLSQRFPKAEFAEVSKLVTDLTKVHTECCHGDLLECADDRADLAKYICENQDSI
SSKLKECCEKPLLEKSHCIAEVENDEMPADLPSLAADFVESKDVCKNYAEAKDVFLGM
FLYEYARRHPDYSVVLLLRLAKTYETTLEKCCAAADPHECYAKVFDEFKPLVEEPQNLI
KQNCELFEQLGEYKFQNALLVRYTKKVPQVSTPTLVEVSRNLGKVGSKCCKHPEAKRM
PCAEDYLSVVLNQLCVLHEKTPVSDRVTKCCTESLVNRRPCFSALEVDETYVPKEFNAE
TFTFHADICTLSEKERQIKKQTALVELVKHKPKATKEQLKAVMDDFAAFVEKCCKADD
KETCFAEEGKKLVAASQAALGLEQKLISEEDLNSAVDHHHHHH
SEQ ID No. 2:
α4-1BB scFv
EFEMQLVESGGGLVQPGRSMKLSCAGSGFTLSDYGVAWVRQAPKKGLEWVAYIS
YAGGTTYYRESVKGRFTISRDNAKSTLYLQMDSLRSEDTATYYCTIDGYGGYSGSHWY
FDFWGPGTMVTVSSGGGGSGGGGSGGGGSDIQMTQSPSLLSASVGDRVTLNCRTSQNV
YKNLAWYQQQLGEAPKLLIYNANSLQAGIPSRESGSGSGTDFTLTISSLQPEDVATYFCQ
QYYSGNTFGAGTNLELKGGSGG
SEQ ID No. 3:
αCD19 scFv-HSA
EFDIQMTQSPASLSTSLGETVTIQCQASEDIYSGLAWYQQKPGKSPQLLIYGASDLQ
DGVPSRFSGSGSGTQYSLKITSMQTEDEGVYFCQQGLTYPRTFGGGTKLELKGGGGSGG
GGSGGGGSEVQLQQSGAELVRPGTSVKLSCKVSGDTITFYYMHFVKQRPGQGLEWIGRI
DPEDESTKYSEKFKNKATLTADTSSNTAYLKLSSLTSEDTATYFCIYGGYYFDYWGQGV
MVTVSSGGSGGAAARGVFRREAHKSEIAHRYNDLGEQHFKGLVLIAFSQYLQKCSYDE
HAKLVQEVTDFAKTCVADESAANCDKSLHTLFGDKLCAIPNLRENYGELADCCTKQEP
ERNECFLQHKDDNPSLPPFERPEAEAMCTSFKENPTTFMGHYLHEVARRHPYFYAPELL
YYAEQYNEILTQCCAEADKESCLTPKLDGVKEKALVSSVRQRMKCSSMQKFGERAFKA
WAVARLSQTFPNADFAEITKLATDLTKVNKECCHGDLLECADDRAELAKYMCENQATI
SSKLQTCCDKPLLKKAHCLSEVEHDTMPADLPAIAADFVEDQEVCKNYAEAKDVFLGT
FLYEYSRRHPDYSVSLLLRLAKKYEATLEKCCAEANPPACYGTVLAEFQPLVEEPKNLV
KTNCDLYEKLGEYGFQNAILVRYTQKAPQVSTPTLVEAARNLGRVGTKCCTLPEDQRL
PCVEDYLSAILNRVCLLHEKTPVSEHVTKCCSGSLVERRPCFSALTVDETYVPKEFKAET
FTFHSDICTLPEKEKQIKKQTALAELVKHKPKATAEQLKTVMDDFAQFLDTCCKAADK
DTCFSTEGPNLVTRCKDALANLEQKLISEEDLNSAVDHHHHHH
SEQ ID No. 4:
αPDL1 scFv-HSA
EFSYVLTQPPSVSVAPGQTARITCGGNNIGSKSVHWYQQKPGQAPVLVVYDDNDR
PSGLPERFSGSNSGNTATLTISRVEAGDEADYYCQVWDSSSDHVVFGGGTKLTVLGGG
GSGGGGSGGGGSEVQLLEPGGGLVQPGGSLRLSCEASGSTFSTYAMSWVRQAPGKGLE
WVSGFSGSGGFTFYADSVRGRFTISRDSSKNTLFLQMSSLRAEDTAVYYCAIPARGYNY
GSFQHWGQGTLVTVSSGGGGSDAHKSEVAHRFKDLGEENFKALVLIAFAQYLQQCPFE
DHVKLVNEVTEFAKTCVADESAENCDKSLHTLFGDKLCTVATLRETYGEMADCCAKQ
EPERNECFLQHKDDNPNLPRLVRPEVDVMCTAFHDNEETFLKKYLYEIARRHPYFYAPE
LLFFAKRYKAAFTECCQAADKAACLLPKLDELRDEGKASSAKQRLKCASLQKFGERAF
KAWAVARLSQRFPKAEFAEVSKLVTDLTKVHTECCHGDLLECADDRADLAKYICENQ
DSISSKLKECCEKPLLEKSHCIAEVENDEMPADLPSLAADFVESKDVCKNYAEAKDVFL
GMFLYEYARRHPDYSVVLLLRLAKTYETTLEKCCAAADPHECYAKVFDEFKPLVEEPQ
NLIKQNCELFEQLGEYKFQNALLVRYTKKVPQVSTPTLVEVSRNLGKVGSKCCKHPEA
KRMPCAEDYLSVVLNQLCVLHEKTPVSDRVTKCCTESLVNRRPCFSALEVDETYVPKEF
NAETFTFHADICTLSEKERQIKKQTALVELVKHKPKATKEQLKAVMDDFAAFVEKCCK
ADDKETCFAEEGKKLVAASQAALGLEQKLISEEDLNSAVDHHHHHH
SEQ ID NO. 5:
αPD1 scFv
EFQVQLVESGGGVVQPGRSLRLSCAASGFTFSSYGMHWVRQAPGKGLEWVAVIW
YDGSNKYYADSVKGRFTISRDNSKNTLYLQMNSLRAEDTAVYYCASNGDHWGQGTLV
TVSSGGGGSGGGGSGGGGSEIVMTQSPATLSVSPGERATLSCRASQSVSSNLAWYQQKP
GQAPRLLIYGASTRATGIPARFSGSGSGTEFTLTISSLQSEDFAVYYCQQYNNWPRTFGQ
GTKVEIKGGSGG
SEQ ID NO. 6:
αTIGIT scFv-HSA
EFDIVMTQSPDSLAVSLGERATINCKSSQTVLYSSNNKKYLAWYQQKPGQPPNLLI
YWASTRESGVPDRESGSGSGTDFTLTISSLQAEDVAVYYCQQYYSTPFTFGPGTKVEIKG
GGGSGGGGSGGGGSEVQLQQSGPGLVKPSQTLSLTCAISGDSVSSNSAAWNWIRQSPSR
GLEWLGKTYYRFKWYSDYAVSVKGRITINPDTSKNQFSLQLNSVTPEDTAVFYCTREST
TYDLLAGPFDYWGQGTLVTVSSGGGGSDAHKSEVAHRFKDLGEENFKALVLIAFAQYL
QQCPFEDHVKLVNEVTEFAKTCVADESAENCDKSLHTLFGDKLCTVATLRETYGEMAD
CCAKQEPERNECFLQHKDDNPNLPRLVRPEVDVMCTAFHDNEETFLKKYLYEIARRHP
YFYAPELLFFAKRYKAAFTECCQAADKAACLLPKLDELRDEGKASSAKQRLKCASLQK
FGERAFKAWAVARLSQRFPKAEFAEVSKLVTDLTKVHTECCHGDLLECADDRADLAK
YICENQDSISSKLKECCEKPLLEKSHCIAEVENDEMPADLPSLAADFVESKDVCKNYAEA
KDVFLGMFLYEYARRHPDYSVVLLLRLAKTYETTLEKCCAAADPHECYAKVFDEFKPL
VEEPQNLIKQNCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCFSALEVDETY
VPKEFNAETFTFHADICTLSEKERQIKKQTALVELVKHKPKATKEQLKAVMDDFAAFVE
KCCKADDKETCFAEEGKKLVAASQAALGLEQKLISEEDLNSAVDHHHHHH
SEQ ID NO. 7: T7 primer
TAATACGACTCACTATAGG
SEQ ID NO. 8: BGH primer
TAGAAGGCACAGTCGAGG
SEQ ID NO. 9: MSA F
gtccccagtcatcagctcctaggggtgtgtttcgccgag
SEQ ID NO. 10: MSA R
GACtctagaggctaaggcgtctttgcatct

### Materials and Sources Used in the Examples

Fusion protein α4-1BB scFv-MSA: expressed by recombinant yeast and purified by AKTA protein purification instrument.

Fusion protein αPD1 scFv-MSA: expressed by recombinant yeast and purified by AKTA protein purification instrument.

Fusion protein αPDL1 scFv-MSA: expressed by recombinant yeast and purified by AKTA protein purification instrument.

Fusion protein αPD-L1 scFv-HSA: expressed by recombinant HEK293T cells and purified by AKTA protein purification instrument.

Fusion protein αEGFR scFv-HSA: expressed by recombinant HEK293T cells and purified by AKTA protein purification instrument.

Fusion protein αHER2 scFv-HSA: expressed by recombinant HEK293T cells and purified by AKTA protein purification instrument.

Fusion protein αTIGIT scFv-HSA: expressed by recombinant HEK293T cells and purified by AKTA protein purification instrument.

B16-F10-OVA cells: constructed in the laboratory.

Poly (lactic acid) PLA137K, molecular weight 137000 Da, ester terminated poly(L-lactide): purchased from Jinan Daigang Biotechnology Co., Ltd.

Dichloromethane: purchased from Guangzhou Chemical Reagent Factory.

Anhydrous ethanol: purchased from China National Pharmaceutical Group Chemical Reagent Co., Ltd.

Protein free blocking solution: purchased from Shanghai Shenggong Biotechnology Co., Ltd.

His-tag antibody (HRP, mouse antibody): purchased from Beijing Yiqiao Shenzhou Biotechnology Co., Ltd.

ELISA colorant: purchased from Beijing Yiqiao Shenzhou Biotechnology Co., Ltd.

PD-L1 antigen: purchased from Beijing Yiqiao Shenzhou Biotechnology Co., Ltd.

Sheep-anti-rat IgG antibody HRP: purchased from Beijing Yiqiao Shenzhou Biotechnology Co., Ltd.

Polystyrene board used in ELISA: purchased from Corning Company in the United States.

IFN ELISA kit: purchased from Shenzhen Dakewei Biotechnology Co., Ltd.

ANNEXIN V Cell Apoptosis Kit: purchased from Jiangsu Kaiji Biotechnology Co., Ltd.

The experimental instrument and models used in the examples:

Ultrasonic cell crusher: VCX130, Sonics, USA.

Rotary evaporator: RV 10 digital V digital display, IKA, Germany.

Microchannel reactor: 1300 SERIES A2, Corning Corporation, USA.

Nanoparticle size and Zeta potentiometers: Nano ZSE, Malvern, UK.

Desktop micro freezing centrifuge: Microfuge 20R, Beckman Coulter, USA.

Microplate reader: BioTek Company, USA.

### Embodiment 1: Design of Single-Chain Antibody Fragment Fusion Protein Gene

Enter the IMGT database, find the antibody sequence search entry, input the antibody target to be queried, select the antibody, enter the amino acid sequence interface, select the VH and VL Kappa or Lamda segment, and connect the two segments with (GGGGS)₃ to obtain the corresponding single-chain antibody fragment peptide sequence.

Wherein, the structures of αHER2 scFv and α4-1BB scFv were VL-L-VH, and the structures of αPDL1 scFv and αTIGIT scFv were VH-L-VL.

The single-chain antibody fragment and the peptide sequence of HSA or MSA without signal peptide were connected through (GGGGS)₃ to obtain the corresponding single-chain antibody fragment-albumin recombinant fusion protein peptide sequence.

The nucleotide sequence of the peptide was converted through SnapGene or other similar software, which was synthesized by Shanghai Shenggong Company.

αHER2 scFv-HSA was constructed by the above-mentioned method, and its sequence is shown in SEQ ID No. 1;

α4-1BB scFv was constructed by the above-mentioned method, and its sequence is shown in SEQ ID No. 2;

αCD19 scFv-HSA was constructed by the above-mentioned method, and its sequence is shown in SEQ ID No. 3;

αPDL1 scFv was constructed by the above-mentioned method, and its sequence is shown in SEQ ID No. 4;

αTIGIT scFv-HSA was constructed by the above-mentioned method, and its sequence is shown in SEQ ID No. 5.

### Embodiment 2: Amplification of MSA Sequence from a Mouse cDNA Library

MSA (Mouse Serum Albumin) cDNA without signal peptide was obtained by PCR amplification from mouse liver fetal cDNA library. Primer MSA F(SEQ ID NO. 8) and MSA R(SEQ ID NO. 9) was synthesized with an oligonucleotide synthesizer with downstream primers introducing XbaI cleavage sites and protective bases. The line is the endonuclease recognition sequence.

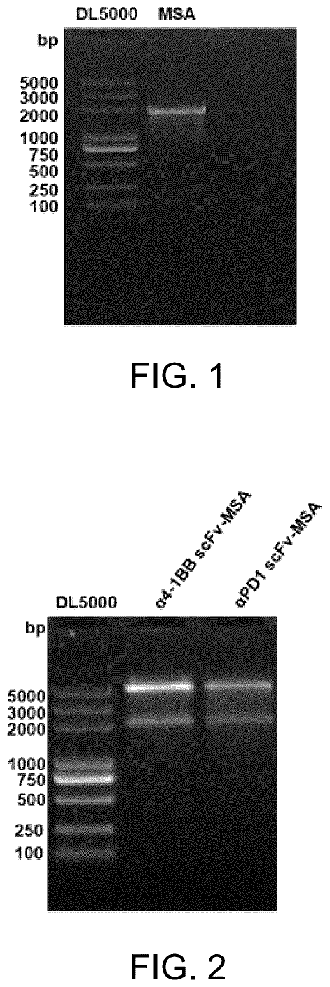

50 μL of PCR reaction system: 25 μL of 2×Mix, DNA template <200 ng, 1 μL of Primer mFcγRT-F (10 pmol/μL), of MSA F(SEQ ID NO. 8)(10 pmol/μL) and 1 μL of MSA R (SEQ ID NO. 9)(10 pmol/μL), make up the rest with ddH₂O supplement, and the reaction system can be scaled down or enlarged according to demand. After gentle mixing, PCR was performed, preheating at 94° C. for 1 min followed by denaturation at 94° C. for 30 s, annealing at 57° C. for 30 s, extension at 72° C. for 1.5 min, a total of 30 cycles; then extension at 72° C. for 5 minutes. The expected 1.6 kb band was obtained through the detection and analysis of 1% agarose gel, as shown in FIG. 1. The gel was recovered and quantified.

### Embodiment 3: Construction of scFv-MSA Yeast System Expression Plasmid by Double Enzyme Digestion

The sequence of α4-1BB scFv was synthesized by Shenggong Company, as shown in SEQ ID No. 2; and the sequence of αPD1 scFv was synthesized by Shenggong Company, as shown in SEQ ID No. 6.

The recombinant fusion protein gene sequence and pcDNA3.1 vector were digested by EcoR I and Xho I, and 50 μL of enzyme digestion reaction system: 1 μg of recombinant fusion protein gene fragment and Plasmid pPICZα, 1 μL of each EcoR I and Xho I, 5 μL of CutSmart buffer with the rest filled with ddH₂O supplement. Enzymatic digestion was performed at 37° C. for more than 2 hours (preferably overnight without star activity), then inactivated by heated at 65° C. for 20 minutes. It was separated by agarose gel electrophoresis, the electrophoresis results are shown in FIG. 2; and the target strip in the gel was cut and the gel was recovered. Insertion fragments and plasmids recovered was connected by T4 DNA ligase, 20 μL-linked reaction system: 2 μL of T4 Reaction Buffer, 10 ng DNA of pcDNA3.1, 40 ng DNA of recombinant fusion protein, 10 μL of ddH₂O, 1 μL of T4 DNA Ligase. The system reacted at 25° C. for 20 minutes or stay overnight at 16° C. for ligation.

### Embodiment 4: Transformation and Identification of Expression Plasmid of Single-Chain Antibody Fragment Albumin Recombinant Fusion Protein into Escherichia coli

Escherichia coli receptive cell DH5a was thawed on ice. Took 50 μL of DH5a and 20 μL of connected plasmid, gently blew and mixed well, then left on ice for 30 minutes; after 45 seconds of heat shock in a water bath at 42° C., immediately transferred to ice and let it stand for 2 minutes; added 200 μL of non-resistant Lysogeny broth medium to the centrifuge tube, resuscitated at 37° C., 200 rpm for 1 hour; then took 20 μL of bacterial solution to coat on LB plates containing ampicillin resistance and incubated upside down at 37° C. for 12 hours.

PCR program was used to verify the construction of expression plasmid of single-chain antibody fragment albumin recombinant fusion protein. The primer sequence was shown in SEQ ID No. 6. After PCR identification, it was characterized by nucleic acid gel electrophoresis, as shown in FIG. 2. The colonies containing the target gene identified by PCR were sent to Shenggong Biotechnology Co., Ltd. for plasmid sequencing, and the sequencing results were consistent with expectations.

### Embodiment 5: Transformation of Recombinant of Single-Chain Antibody Fragment Albumin Recombinant Fusion Protein in Yeast

Plasmid DNA was linearized and dephosphorylated. 50 μL of enzyme digestion reaction system was 5 μg of plasmid DNA, 5 μL of CutSmart Buffer (10×), 1 μL of PmeI, 1 μL of Quick CIP, adding ddH₂O supplement to 50 μL. Enzyme digestion was performed at 37° C. for more than 2 hours on a PCR instrument, and stopped by heat inactivation at 65° C. for 20 minutes.

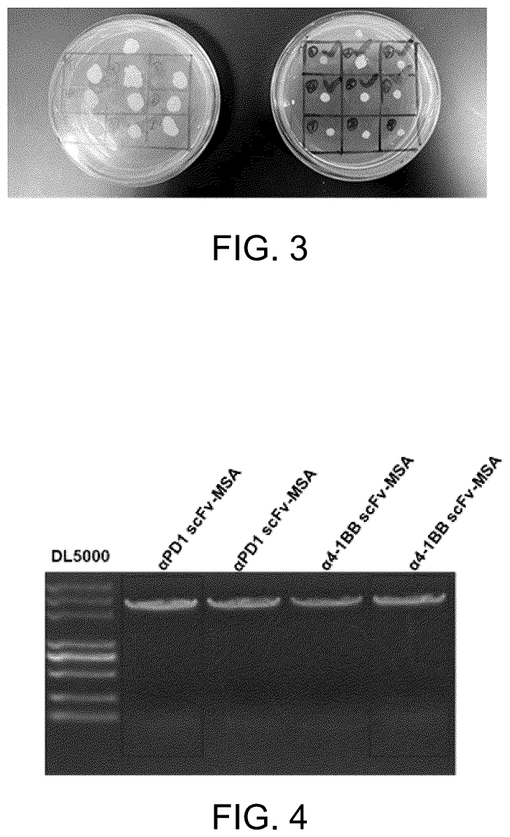

A tube of competent cells was thawed at room temperature and added 3 μL linearized DNA vector. 1 mL of solution II was added to the DNA/cell mixture and mixed through a vortex or light bomb centrifuge tube. The conversion mixture was incubated in a water bath or incubator at 30° C. for 1 hour, mixing with vortex or light bombing centrifuge tubes every 15 minutes for the conversion reactions. The cells were heat shocked in a hot block or water bath at 42° C. for 10 minutes, then divided into two tubes (approximately 525 μL each tube) and added 1 mL of YPD culture medium into each tube. The cells were incubated at 30° C. for 1 hour to express the Zeocin resistance gene, then were pelleted by centrifugation at room temperature at 3000×g for 5 minutes, discarding the supernatant. Each tube of cells was resuspended with 500 μL of solution III and the two tubes of cells were combined into one tube. The cells were pelleted by centrifugation at room temperature at 3000×g for 5 minutes, discarding the supernatant. The cells were resuspended with 100-150 μL of solution III. All transformant was screened on an appropriate plate using a sterile applicator. The plate was incubated at 30° C. for 3 to 10 days, and each transformation should produce approximately 50 colonies. Mut+ positive clones were screened by simultaneously inoculating colonies on MDH (glucose as carbon source) and MMH (methanol as carbon source) plates, and by comparing the differences in colony size. As shown in FIG. 3, colonies 1, 2, 3, 4, 5, and 6 did not show significant differences in growth on MDH and MMH plates, so they were considered as Mut+ positive clones.

6-10 zeocin resistant Pichia pastoris transformants were selected and the presence of inserts was analyzed using PCR, see FIG. 4.

### Embodiment 6: Induced Expression of Recombinant Yeast with Mut⁺ (Shake Flask Culture)

A single colony was selected and placed in a 250 mL shake flask filled with 25 mL of BMGY culture medium, incubated at 28-30° C., 250-300 rpm until OD600=2-6 (16-18 hours). Took 1 mL of culture medium and froze it; the remain was centrifuged at 1500-3000 g at room temperature for 5 minutes, collected the bacterial, resuspended with BMMY to make OD600=1.0 (about 100-200 mL), and then started to induce expression; the obtained bacterial solution was placed in a 1 L shaker flask, sealed it with double-layer gauze or coarse cotton cloth, and placed it in a shaker at 20-30° C. with a rotational speed of 250-300 rpm to continue growing; 100% methanol was added into the culture medium every 24 hours to a final concentration of 0.5-1.0%; 1 mL of bacterial liquid samples was taken at different time points, and placed in a 1.5 mL EP tube. The tube was centrifuged at maximum speed for 2-3 minutes; the supernatant and bacterial cells were collected respectively to analyze the expression level of the target protein and the optimal harvesting time of the bacterial liquid. The time points are generally taken as 0, 6, 12, 24, 36, 48, 72, 84, and 96 hours.

### Embodiment 7: Purification and Characterization of the Expression Product of Recombinant Yeast with Mut⁺

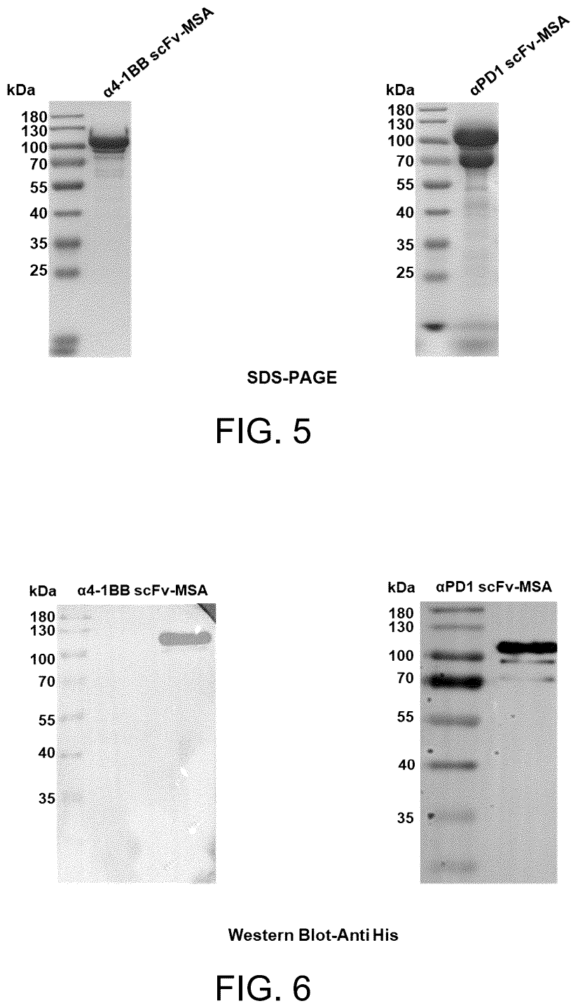

Recombinant yeast expression broth was centrifuged at 8000 g for 20 minutes and the supernatant was collected. After the supernatant was concentrated to a suitable volume by a tangential flow equipment, the culture medium and nickel column preservation solution were replaced by Binding Buffer (Tripotassium phosphate buffer). The fermentation product was mixed with a nickel column and incubated in a refrigerator at 4° C. The nickel column was washed 5 times with a Washing Buffer (low concentration imidazole) to remove the impurities. The packing was resuspended with Elution Buffer (high concentration imidazole) and left to stand for 20 minutes. When the packing was settled at the bottom of the tube, collected the effluent. For a 1 L of fermentation system, an Akta protein purification system was often used for protein purification. Akta is a preparative protein purification chromatography system used for rapid and safe amplification of protein purification and separation. The separation process included: 1) balance: using Binding buffer (Tripotassium phosphate buffer) to balance the nickel column; 2) binding: loading samples using a sample cup or an automatic sample pump and the sample flow rate was controlled at 0.5 to 1 mL/min; 3) washing: the nickel column with 10 nickel columns was washed with a phosphate buffer containing 20 mM imidazole with a sample flow rate controlled at 2 mL/min; 4) washing out: the nickel column with 10 column volumes was washed out with a phosphate buffer containing 500 mM imidazole with a sample flow rate controlled at 2 mL/min. The eluate was collected, concentrated by using a 10 kDa ultrafiltration tube, and the purity of the protein was characterized by SDS-PAGE, as shown in FIG. 5.

Anti-His-HRP antibodies were used to conduct WB experiments to detect the expression of the target protein, as shown in FIG. 6.

### Embodiment 8: Mammalian Expression System Expressing Recombinant Fusion Proteins

Plasmids αHER2 scFv-HSA-pcDNA3.1, αEGFRVIII scFv-HSA-pcDNA3.1, and αTIGIT scFv-HSA-pcDNA3.1 were synthesized by Shenggong Company. 18 μg of plasmid was mixed with serum-free F-12K culture medium to 500 μL. 54 μL of PEI was mixed with serum-free F-12K medium to 500 μL. The F12-K/PEI mixture was added dropwise to the plasmid mixture, mixed evenly, and incubated at room temperature for 10 minutes while flicking the EP tube gently. After incubation, the mixture was mixed with approximately 1×10⁷ CHO-K1 or HEK 293T cells and cultured at 37° C. for 6-8 hours. Then the medium was replaced with serum-free F-12K medium and cultured for 72 hours.

### Embodiment 9: Purification of Fusion Protein Expressed in Mammalian Expression System

Nickel column purification belongs to Immobilized Zirconium Ion Affinity Chromatography (IMAC), which has high loading capacity, high purification speed, and high specificity. Transition metals can purify specific proteins by forming stable chelation with carboxyl or amino groups of amino acids. The elution of Ni column adopted competitive elution. First, low concentration imidazole was used to remove impurities and poorly bound proteins, and then an appropriate concentration of imidazole was used to elute the target protein.

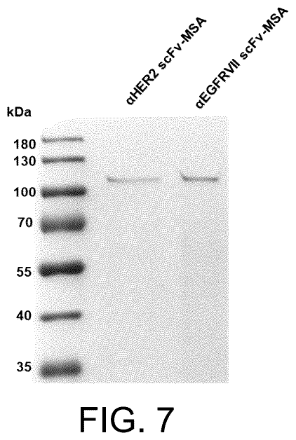

After 72 hours of transfection, the supernatant was collected and concentrated to a suitable volume, and then the culture medium and nickel column preservation solution were replaced with Binding Buffer (Tripotassium phosphate buffer). The fermentation product was mixed with a nickel column and incubated in a refrigerator at 4° C. The nickel column was washed for 5 times with a Washing Buffer (low concentration imidazole) to wash out the impurities. The packing was resuspended with Elution Buffer (high concentration imidazole) and left to stand for 20 minutes. When the packing was settled at the bottom of the tube, the effluent was collected and concentrated with an ultrafiltration tube. The protein produced was characterized by SDS-PAGE, and the results are shown in FIG. 7. After purification of the recombinant fusion protein, a clear band appeared at the molecular weight position of 100 kDa, indicating that the molecular weight of the recombinant fusion protein was around 110 kDa.

### Embodiment 10: Characterization of the Structures of the Recombinant Fusion Proteins Expressed in a Mammalian System

(1) Structural Characterization

The purified recombinant fusion proteins were diluted 10 times, 100 times, and 1000 times respectively, and 20 μL of each recombinant fusion protein of different dilution concentrations was taken to detect protein sample concentration by BCA method.

Based on the concentration of the recombinant fusion protein, took a 20 ng sample and mixed it with 5×SDS-Loading buffer at a ratio of 4:1. Incubated at 99° C. for 15 minutes. Prepared 10% polyacrylamide gel, put 20 ng sample on each well, and conducted 80V electrophoresis for 2 hours.

The gel was removed and placed in the tank containing membrane transfer solution. In the Petri dish with transfer solution, put the cut glue, PVDF membrane soaked with ethanol, filter paper and sponge, let it balance for 5 minutes; placed the rotating film clamp horizontally with the black side on the bottom and the transparent side on the top. Stacked the following in sequence: sponge, four pieces of filter paper soaked in the transfer solution, PVDF membrane, gel after electrophoresis, four pieces of filter paper soaked in the buffer solution and sponge. Used a pipette to roll over the stacked filter paper to remove air bubbles. Installed the transfer clamp in the transfer frame, placed it in the electrophoresis tank, added pre-cooled transfer solution, and placed an ice box of appropriate size in the gap of the electrophoresis tank. Connected the electrophoresis tank and electrophoresis instrument, and performed electrophoresis in constant current mode at 300 mA for 100 minutes.

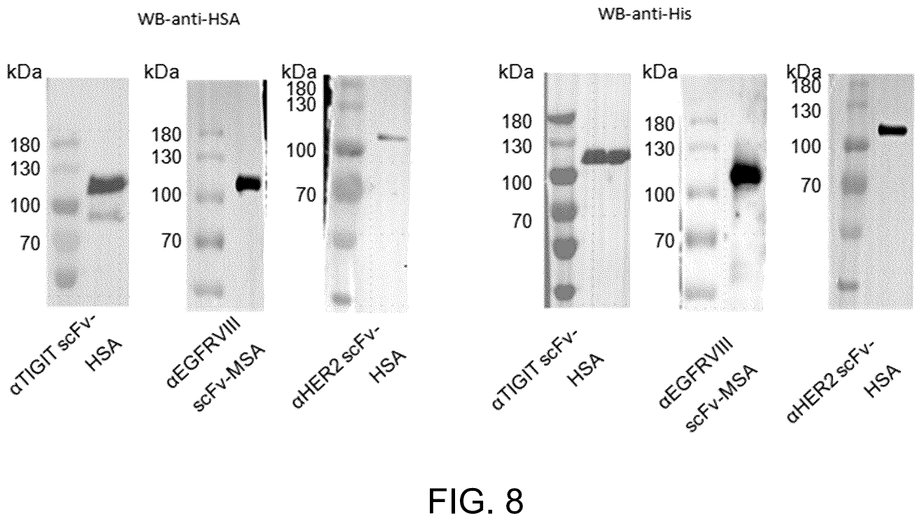

Moved the PVDF membrane to a petri dish containing blocking solution (4% BSA, prepared with PBST), and shook it on a decolorization shaker at room temperature to block for 2 hours; diluted the primary antibody with a diluent (1% BSA, prepared with PBST) to an appropriate concentration (according to the antibody specification), and added the primary antibody dilution into a Petri dish and incubated at room temperature for 2 hours, washed with PBST on a decolorizing shaker at room temperature for three times, each time for 10 minutes; added the secondary antibody diluent (1:5000, 1% BSA, prepared with PBST) into the Petri dish, placed it on the shaker, incubated at room temperature for 45 minutes; then washed it with PBST on a decolorizing shaker at room temperature for three times, each time for 10 minutes. Added developing solution for development. The results were shown in FIG. 6 and FIG. 8, in which the recombinant fusion protein had consistent molecular weight Western blotting under the primary antibody labeling of His, HSA, and Protein L, indicating that the recombinant fusion protein had three structures: His, HSA, and scFv.

### Embodiment 11: Characterization of the Affinity of Recombinant Fusion Proteins Expressed in Mammalian Systems

(2) Affinity Characterization

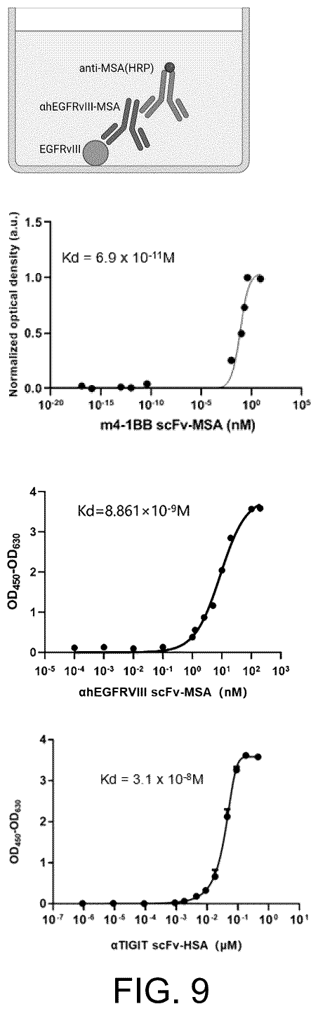

The protein affinity of the purified product was characterized by ELISA. Human EGFRvIII, or human TIGIT, or mouse 4-1BB antigen was diluted to 1 μg/mL with Coating buffer; added 100 μL/well and incubated at 37° C., then at 4° C. overnight for coating, and then washed with PBST for three times; blocked with 5% BSA, 200 μL/well, and then washed with PBST for three times. Protein αhEGFRvIII scFv-MSA or αhTIGIT scFv-HSA or α4-1BB scFv-MSA was quantified by BCA method, and diluted with Ultrapure water to 20 μg/mL, 10 μg/mL, 2 μg/mL, 1 μg/mL, 0.5 μg/mL, 0.25 μg/mL, 0.125 μg/mL, 0.1 μg/mL, 0.01 μg/mL, 0.001 μg/mL, 0.0001 μg/mL, 0.00001 μg/mL, added sample with 100 μL/well, incubated at 37° C. for 2 hours, then washed with PBST for six times. Anti-Mouse-Serum-Albumin-Antibody (HRP) was used as a secondary antibody, washed with PBST for six times. ELISA chromogenic solution was used and reacted in a dark environment for 10 minutes, and then the reaction was terminated with 2M sulfuric acid. The absorbance of OD450-OD630 was measured by a Microplate reader, and the results are shown in FIG. 9.

### Embodiment 12: Preparation of Recombinant Fusion Protein Nano-Assembly

The recombinant fusion protein of the purified α4-1BB-MSA, αPD1-MSA and αPDL1-MSA single-chain antibody fragment (quantified by Nanodrop One ultra-micro UV spectrophotometer to determine the concentration) were mixed at 1:1:1. Prepared a solution of the total protein concentration of 5 mg/mL and a chloroform solution of 5 mg/mL polylactic acid (PLA₁₃₇ₖ) with Ultrapure water. Took 1 mL of 5 mg/mL single-chain antibody fragment recombinant fusion protein aqueous solution into a 15 mL centrifuge tube, added 100 μL of 5 mg/mL polylactic acid (PLA₁₃₇ₖ) chloroform solution (i.e. the mass ratio of the recombinant fusion protein of the single-chain antibody fragment to PLA₁₃₇ₖ is 10:1), then ultrasonic emulsification was performed by an ultrasonic cell disruption instrument in an ice water bath, wherein the ultrasonic power was 130 W, the amplitude was 50%, the ultrasound time was 1.5 minutes, and after 5 seconds of ultrasound, stopped for 2 seconds (the interruption time is not included in the ultrasound time). After ultrasound emulsification, the emulsion was transferred to a 100 mL round-bottomed flask, washed out the residual emulsionin the centrifuge tube with ultrapure water, and transferred the washing solution into the 100 mL round-bottomed flask. The eluent was rotary evaporated in a Rotary evaporator sequentially according to the vacuum degree of 200/100/50/30/20 mbar, holding for 10 minutes under each vacuum degree. When the vacuum degree was 30/20 mbar, immersed the round-bottomed flask in a 32° C. water bath to fully remove chloroform and evaporate a certain volume of water to concentrate the volume of the nanoparticle solution. After the rotary evaporation, nanoparticles of single-chain antibody fragments-recombinant fusion protein fusion protein-polylactic acid were collected for later use.

### Embodiment 13: Particle Size Characterization of Recombinant Fusion Protein Nano-Assembly of Single-Chain Antibody Fragments

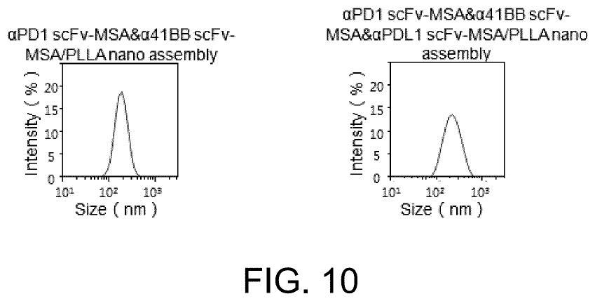

100 μL of the purified resuspended particle solution in Embodiment 13 was placed in a particle size pool, and the hydration diameter of the nanoparticles was measured by a nanoparticle size analyzer and a Zeta potential analyzer. The measured particle size of the nanoparticles was around 100-200 nm corresponding to α4-1BB/αPD1/αPDL1 nano-assembly, and its particle size distribution diagram is shown in FIG. 10 with the particle size being 200.1 nm and the PDI dispersion being 0.19 indicating good uniformity. The particle size distribution diagram of the nano-assembly of α4-1BB/αPD1 is shown in the left figure of FIG. 10 with the particle size being 182.6 nm and the PDI dispersion being 0.15 indicating good uniformity.

### Embodiment 14: Morphological Characterization of Nano-Assembly of Recombinant Fusion Protein of Single-Chain Antibody Fragments

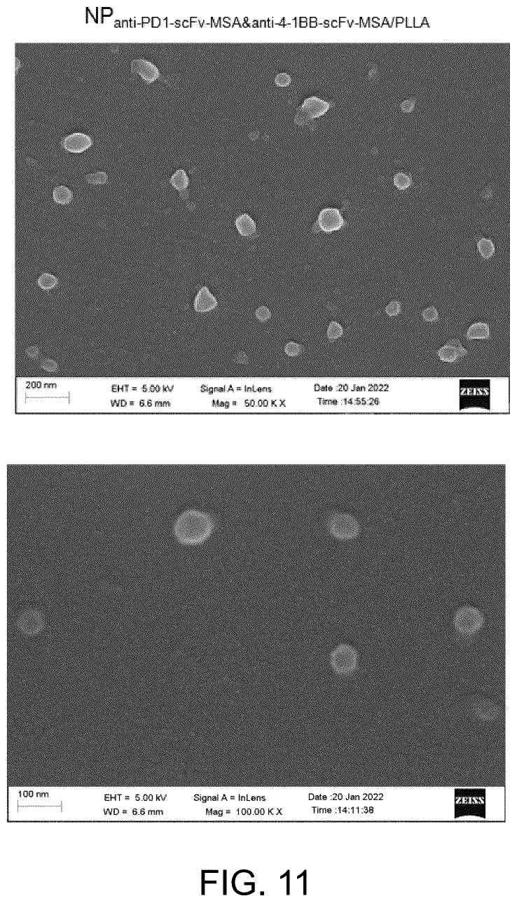

The nano-assembly of α4-1BB/αPD1 was centrifuged at low speed (400 rcf, 4° C.) for 5 minutes using a desktop micro freezing centrifuge to remove the unassembled insoluble polyester. The supernatant was transferred to a new EP tube and centrifuged at high speed (15000 rpm, 4° C.) for 60 minutes to separate the free protein and nano-assembly. The free fusion protein in the supernatant was removed, and the precipitation of the lower assembly was resuspended with 1×PBS, and the concentration of the purified nanoassembly was diluted to 0.1 mg/mL, and took 2 μL and dropped it onto the silicon wafer and observed under a scanning electron microscope after 8 hours of water evaporation. As shown in FIG. 11, the nano-assembly was an independently existing spherical shape.

### Embodiment 15: Activation of T Cell Proliferation by Nano-Assemblies of Recombinant Fusion Protein of Single-Chain Antibody Fragments

After the eyeballs were removed and bloodletting, the OT1 mice's the carotid artery was cut open to allow sufficient bloodletting, and the spleen was removed.

The spleen was ground with a syringe handle (assisted by nylon mesh), the grinding solution was filtered through a 200 mesh nylon mesh into a 15 mL centrifuge tube, centrifugated at 450 g for 5 minutes to collect cells.

According to the needs of the experimental, cells to be purified were transferred into a mL centrifuge tube, centrifuged at 300 g for 10 minutes, discarded the supernatant, and collected the cells.

Every 1×10⁷ cells were added 5 μL of anti-CD8a micro beads, labeled at 4° C. for 15 minutes. Filled up with MACS buffer to wash and remove unlabeled micro beads, then centrifuged at 300 g for 10 minutes (450 g or 800 g for 5 minutes was also acceptable). Discarded the supernatant and collected the cells. The cells were resuspended with MACS buffer in a volume of 1×10⁸ cells in 500 μL. The sorting column was placed on the sorter, and 2 layers of 200 mesh nylon mesh was placed on the sorting column. The column was moistened with 500 μL of MACS buffer, and collected the eluent with a new 15 ml centrifuge tube underneath the sorting column. The resuspended cells were added to the sorting column through two layers of 200 mesh nylon mesh, and washed with 1.5 mL of MACS buffer in the sorting column. 1 mL of MACS buffer were added to the sorting column, and then the sorting column was removed from the sorter and placed on a 15 mL centrifuge tube. The liquid in the sorting column was quickly pushed into a 15 mL centrifuge tube by a matching pushed handle.

The CFDA-SE dissolved in DMSO was diluted with 1×PBS to make a 10 μM working fluid. The cell precipitates in Ep tubes were collected and fully resuspended with 500 μL of 1×PBS; added 500 μL of 10 μM CFDA-SE staining buffer, and mixed well quickly. The cells were labeled in the dark in a 37° C. cell incubator for 10 minutes, then washed the cells with PBS containing 10% of FBS, centrifuged at 3000 g for 2 minutes and washed twice. The number of the cells were counted, and samples were prepared.

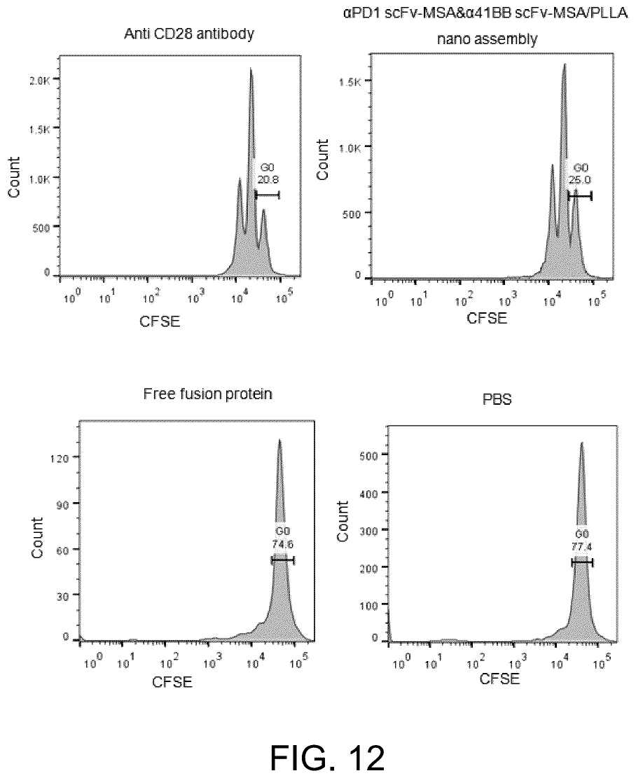

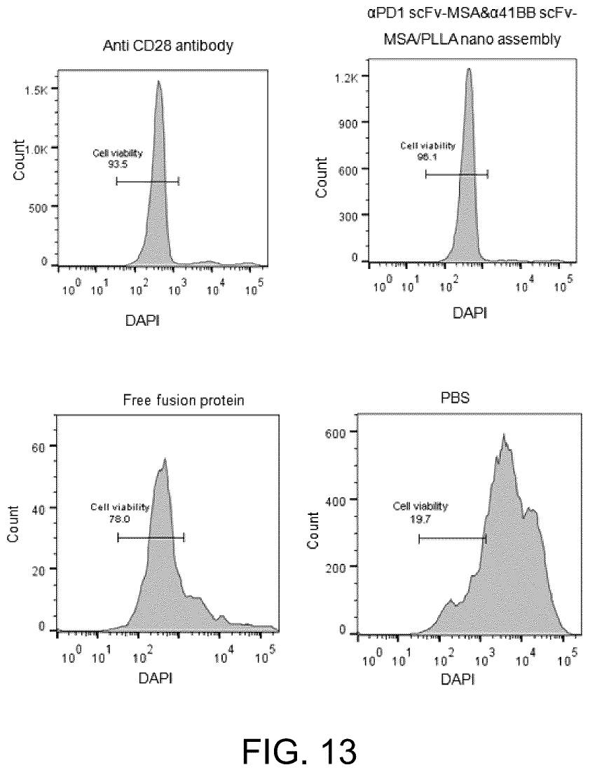

Coating of anti-CD3 antibody: the anti-CD3 antibody was diluted to 5 μg/mL with PBS, then pipetted 60 μL into a round-bottom 96 well plate and incubate at 37° C. for 2 hours. Anti-CD3 antibody was removed and the plate was washed for three times with PBS. 1×10⁶/well of purified CD3⁺T cells were added to a 96-well plate coated with anti-CD3 antibody, and added 1 μg/mL anti-CD28 antibody, or αPD1/α4-1BB nano-assembly, or free αPD1/α4-1BB recombinant fusion protein or PBS to the medium, cultured at 37° C., 5% of CO₂ for 72 hours, then the CFSE fluorescence and DAPI fluorescence of the cells were detected by flow cytometry. The fluorescence results of CF SE are shown in FIG. 12, the experimental group with the addition of αPD1/α4-1BB nano-assembly showed a proliferation peak in T cells, similar to the CD28 antibody positive control group, indicating that αPD1/α4-1BB nano-assembly has the function of activating T cell proliferation. The results of DAPI fluorescence flow cytometry are shown in FIG. 13, the 72-hour survival rate of T cells in the experimental group with the addition of nano-assemblies of αPD1/α4-1BB at was 96%, which was similar to the positive control group of CD28 antibody, the 72-hour survival rate of T cells in the group with PBS was only about 20%.

### Embodiment 16: Nano-Assembly of Recombinant Fusion Protein of Single-Chain Antibody Fragments Enhancing the Killing Effect of T Cells on Tumor Cells

B16-F10-0VA cells were cultivated to a confluence of 80%, added 1 mL of 1×PBS after the supernatant was removed, washed once, and discarded the supernatant. Added 500 μL of 0.25% tryps into to B16-F10-OVA cells, and digested at 37° C. for 2 minutes, then added 2 mL of 1640 complete culture medium to terminate digestion, centrifuged at 800 rpm for 3 minutes, and removed the supernatant.

B16-F10-OVA cells were resuspended with an appropriate volume of 1640 complete culture medium to a density of appropriate of 1×10⁵/mL. Added 500 μL of B16-F10-0VA cell suspension to each well of a 24-well plate, then added IFN-γ to 20 ng/mL, incubated at 37° C. for 24 hours.

OT1-CD3⁺ T cells purified in Embodiment 15 were diluted to 1 million/mL. Took the above-mentioned 24-well plate inoculated with B16-F10-OVA cells, discarded the supernatant, and added 500 μL of OT1-CD3⁺ T cell suspension to each well, then added αPD1/α4-1BB nano-assembly to each well of the experimental group until the concentration of fusion protein was 5 μg/mL, added an equal amount of free fusion protein to each well in the control group, and added a corresponding volume of 1×PBS in the negative control group, and cultured at 37° C. for 24 hours.

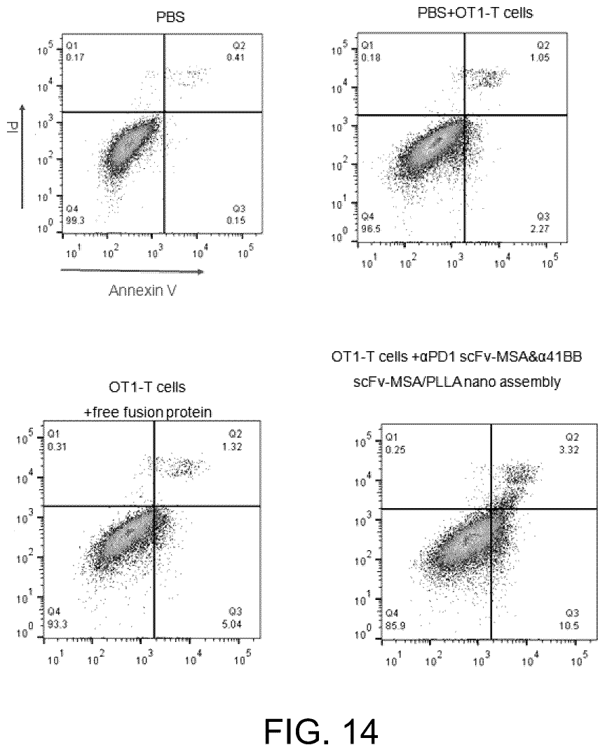

After incubating at 37° C. for 24 hours, discarded the supernatant. The plate was washed for three times with 1 mL of 1×PBS, added 50 μL of 0.25% trypsin to each well, and digested at 37° C. for 2 minutes. 1 mL of 1640 complete culture medium was added to each well to terminate digestion. The plate was centrifuged at 800 rpm for 3 minutes, and disgarded the supernatant and obtained B16-F10-OVA cell precipitate. The cells in each well were resuspended with 500 μL of Binding buffer in Annexin V kit, added 5 μL of Annexin V and 5 μL of PI reagent, fully mixed and incubated at room temperature for 15 minutes, then detected with flow cytometry. The results are shown in FIG. 14, in the experimental group of the αPD1/α4-1BB nano-assembly, there was a significant increase in the proportion of early and late apoptosis of B16-F10-0VA cells, indicating that the αPD1/α4-1BB nano-assembly enhanced the killing effect of OT1-CD3⁺ T cells on B16-F10-0VA tumor cells.

### Embodiment 17: Nano-Assembly of Recombinant Fusion Protein of Single-Chain Antibody Fragments Enhanced T Cell Cytokine Secretion

According to the method described in Embodiment 16, OT1-CD3⁺ T cells were co-cultured with B16-F10-OVA tumor cells, and added αPD1/α4-1BB nano-assembly, free αPD1 scFv-MSA and α4-1BB scFv-MSA fusion protein, and 1×PBS respectively, cultured at 37° C. for 24 hours.

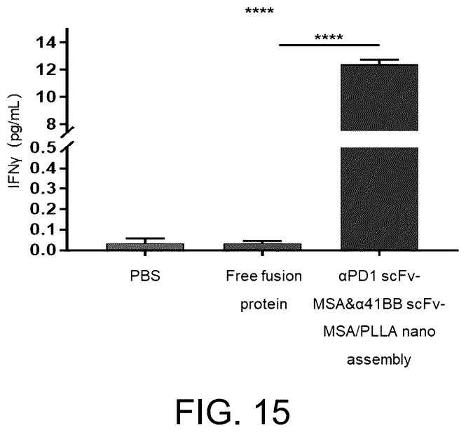

The supernatant was collected and centrifuged at 10000 g for 10 minutes, and collected the supernatant. The obtained culture medium was diluted 10 times and 100 times respectively. IFN-γ in the supernatant was detected by using an IFN-γ detection ELISA kit. The results are shown in FIG. 15. αPD1/α4-1BB nano-assembly significantly enhanced the secretion of cytokines IFN-γ of T cells compared with free fusion proteins.

The technical features of the embodiments above can be combined arbitrarily. To simplify the description, all possible combinations of the technical features of the embodiments above are not described. However, as long as there is no contradiction in the combination of these technical features, they should be considered to be the scope recorded in the description.

The embodiments above express several implementations of the present disclosure only. The description of the embodiments is relatively specific and detailed, but may not therefore be construed as the limitation on the patent scope of the present disclosure. It should be noted that a person of ordinary skill in the art may further make several variations and improvements without departing from the concept of the present disclosure. These variations and improvements all fall within the protection scope of the present disclosure. Therefore, the patent protection scope of the present disclosure shall be defined by the appended claims.

What is claimed is:

1. A nano-assembly, being composed of at least one fusion protein combined with a hydrophobic degradable polyester or its derivative through hydrophobic interaction, wherein the fusion protein comprising a hydrophobic region and an immunoregulatory antibody functional fragment, and the hydrophobic region and the immunoregulatory antibody functional fragment are directly connected or connected through peptide connectors, the immunoregulatory antibody functional fragment is single-chain antibody fragment, and the single-chain antibody fragment has the ability to specifically recognize and bind to an antigen, wherein each of the single chain antibody fragments of each of the at least one fusion proteins are different; the hydrophobic degradable polyester or its derivative is an aliphatic polyester or its derivative with poor water affinity, or an aliphatic polyester or its derivative modified with polyethylene glycol; the fusion protein with the hydrophobic region contains at least 5 hydrophobic structural domains; or the fusion proteins with the intact hydrophobic domains which derived through substitution, deletion, and/or addition of one or more amino acids.

2. The nano-assembly according to claim 1, wherein the single-chain antibody fragment is selected from at least one of the following single-chain antibody fragments derived from humans or other species: CD137L, CD137, 4-1BBL, 4-1BB, OX40L, OX40, ICOSL, ICOS, CD86, CD80, CD28, LFA3, CD2, CTLA4, PDL1, PD1, CD70, CD27, GALS, TIM3, CD111, CD96, CD112, CD226, CD115, CD113, TIGIT, CD39, CD73, CD47, SIRP α, TNF α, IL1 β, IL6, TGF β, IL-10 and IL-12.

3. The nano-assembly according to claim 1, wherein the protein with the hydrophobic region is albumin.

4. The nano-assembly according to claim 3, wherein the albumin is at least one of the following: human serum albumin, mouse serum albumin, bovine serum albumin, rabbit serum albumin, and chicken egg albumin.

5. The nano-assembly according to claim 2, wherein the single-chain antibody fragment is composed of VH and VL, wherein VL is an antibody light chain variable region, peptide or polypeptide sequence, and VH is an antibody heavy chain variable region, peptide or polypeptide sequence, therapeutic protein and its fragments.

6. The nano-assembly according to claim 4, wherein the single-chain antibody fragment is connected to the albumin through a connecting peptide, which is either [GGGGS]n or (EAAAK)n, wherein n is any integer.

7. The nano-assembly according to claim 6, wherein the connecting peptide between the single-chain antibody fragment and the albumin comprises at least one of the following formulas: VL-VH-AC, VL-VH-AN, VH-VL-AC, VH-VL-AN, VL-VH-L-AC, VL-VH-L-AN, VH-VL-L-AC, VH-VL-L-AN, wherein L is the connecting peptide, AC is a C-terminal end of the albumin sequence, and AN is an N-terminal end of the albumin.

8. The nano-assembly according to claim 1, wherein it is assembled from at least two recombinant fusion proteins.

9. The nano assembly according to claim 8, wherein the recombinant fusion protein comprises two or three types.

10. The nano-assembly according to claim 1, wherein the aliphatic polyester is at least one of polylactide, polyglycolide, poly(glycolide-co-lactide) and polycaprolactone.

11. The nano-assembly according to claim 10, wherein the aliphatic polyester is polylactide; the polylactide is poly(L-lactide), poly(R-lactide), or a racemic polylactide; an end group of the polylactide is at least one of ester, carboxyl, and hydroxyl groups.

12. The nano-assembly according to claim 11, wherein the polylactide is a poly(L-lactide), and the end group of the poly(L-lactide) is an ester group.

13. The nano-assembly according to claim 12, wherein a molecular weight range of the poly(L-lactide) is 7200 Daltons to 110000 Daltons.

14. The nano-assembly according to claim 12, wherein the nano-assembly is a nano particle with a particle size ranging from 80 nm to 200 nm.

15. A preparation method of the nano-assembly according to claim 1, comprising the following steps:
(1) mixing the at least one fusion protein according to claim 1 with water or an aqueous solution to obtain a water phase with a concentration of 0.5 mg/mL to 20 mg/mL; and
mixing the hydrophobic degradable polyester or its derivatives with an organic solvent at a concentration of 0.5 mg/mL to 10 mg/mL, to obtain an oil phase; and
(2) preparing the water phase and the oil phase in the step (1) into an oil-in-water emulsion, the volume ratio of the water phase to the oil phase is 1:1 to 10:1; and
(3) separating and purifying the emulsion to obtain a nano-assembly.

16. The preparation method of the nano-assembly according to claim 15, wherein the water phase concentration in the step (1) is 5 mg/mL to 10 mg/mL.

17. The preparation method of nano-assembly according to claim 16, wherein mixing the hydrophobic degradable polyester or its derivatives with the organic solvent in the step (1) at a concentration of 1 mg/mL to 5 mg/mL; and the volume ratio of the water phase to the oil phase is 5:1 to 10:1.

18. The preparation method of the nano-assembly according to claim 15, wherein a weight ratio of the hydrophobic degradable polyester to the fusion protein is 1:0.1 to 1:30.

19. The preparation method of nano-assembly according to claim 17, wherein the organic solvent is at least one of chloroform, dichloromethane, ethyl acetate, methanol and acetonitrile.

20. Therapeutic drugs, comprising an active ingredient includes the nano-assembly according to claim 1, wherein the drugs are tumor immunotherapeutic drugs or therapeutic drugs for autoimmune diseases treatment, or inflammation treatment, or infection treatment.
